(19) United States
(12) Patent Application Publication
(10) Pub. No.: US 2009/0286781 A1
(43) Pub. Date: Nov. 19, 2009

(54) Use of Suluble Guanylate Cyclase Acitvators for Treating Acute and Chronic Lung Diseases

(75) Inventors: Thomas Krahn, Hagen (DE); Johannes-peter Stasch, Solingen (DE); Gerrit Weimann, Köln (DE); Wolfgang Thielemann, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealtHCare AG, Leverkusen (DE)

(21) Appl. No.: 12/083,121

(22) PCT Filed: Sep. 23, 2006
(86) PCT No.: PCT/EP2006/009264

§ 371 Date: Jun. 10, 2009

(30) Foreign Application Priority Data
Oct. 6, 2005 (DE) 10 2005 047 946.4

(51) Int. Cl. A61K 31/5377; C07C 229/34; A61K 31/197; C07D 487/04; A61K 31/506; A61P 9/12; C07D 417/12

(52) U.S. Cl. 514/234.2; 562/444; 514/567; 544/122; 544/328; 514/256; 514/231.5; 544/146

(57) ABSTRACT

The present invention relates to the use of compounds of the formulae I-VI for manufacturing a pharmaceutical for the treatment of acute and chronic lung disorders such as the respiratory distress syndromes [acute lung injury (ALI), acute respiratory distress syndrome (ARDS)] and the treatment of COPD.

The present invention relates to the use of compounds of the formulae I-VI for manufacturing a pharmaceutical for the treatment of acute and chronic lung disorders such as the respiratory distress syndromes [acute lung injury (ALI), acute respiratory distress syndrome (ARDS)] and the treatment of COPD.

Various noxae which directly damage the lung (pneumonias, aspiration, contusion, fat embolisms, inhalation traumas, reperfusion edema) and extrapulmonary noxae (sepsis, massive transfusions, medicament side effects, acute pancreatitis) may induce an ALI or ARDS. According to the 1994 AECCC Definition, when a lung disorder which is associated with bilateral infiltrates in the chest radiograph and seriously impairs gas exchange and requires ventilation occurs acutely after exposure to one of the abovementioned noxae, the term used is ALI when the PaO₂/FiO₂ ratio is ≦300 and is ARDS when the PaO₂/FiO₂ ratio is ≦200. The mortality associated therewith is reported to be above 50% and thus represents a serious intensive care syndrome.

The pathophysiological findings are diffuse alveolar damage with invasion by neutrophils, macrophages, erythrocytes, development of hyaline membranes, emergence of protein-rich edema fluid, and loss of integrity of the alveolar epithelial barrier with pathologically increased permeability. Histology reveals after the stage of pulmonary edema formation an acute and chronic inflammatory reaction with a possible transition to fibrosis. The main clinical feature is a drastic deterioration in gas exchange with reduced oxygenation and impeded ventilation through an impaired ventilation-perfusion distribution. Most ALI/ARDS patients additionally show moderately severe pulmonary hypertension with increased pulmonary resistance, the causes being a hypoxic vasoconstriction and a destruction and obstruction of the pulmonary vascular endothelium. In some ALI/ARDS patients, this may lead to right heart failure.

Previous attempts at therapy to reduce the pulmonary arterial pressure in ALI/ARDS have been carried out with hydralazine, prostaglandin E1 and inhaled NO, in all cases without effect on mortality or reducing the ventilation time.

One of the most important cellular transmission systems in mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triphosphate (GTP). The representatives of this family which are known to date can be divided into two groups both according to structural features and according to the nature of the ligands: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory center. This has a central importance for the mechanism of activation. NO is able to bind to the iron atom of the heme and thus distinctly increase the activity of the enzyme. Heme-free preparations by contrast cannot be stimulated by NO. CO is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes.

It has now surprisingly been found that the activators according to the invention of soluble guanylate cyclase listed below, compounds I-VI, are particularly suitable for producing pharmaceutical substances/medicaments for reducing pulmonary hypertension. Compared with the prior art, the compounds of the invention of the formulae I to VI have improved pharmacodynamic properties: on the one hand they act independently of NO produced endogenously in the pulmonary circulation, even if there is severe endothelial damage and the disease is at an advanced stage, to lower the pressure in the pulmonary arterial circulation. In addition, the stimulators of soluble guanylate cyclase enhance the effect of endogenously produced NO and, in this way, improve the gas exchange through a selective pulmonary vasodilatation of the ventilated areas, leading to a reduction in the intrapulmonary shunt with an increase in oxygenation.

Compound (I) corresponds to the following formula:

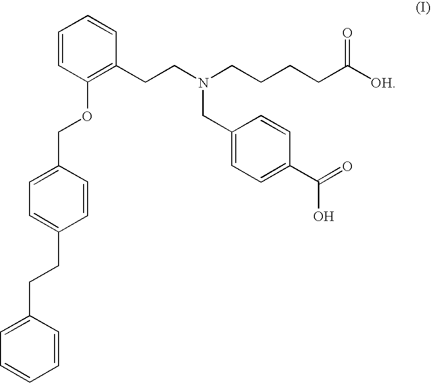

Compound (I), the preparation and use thereof as pharmaceutical have been disclosed in WO 01/19780.

Compound (II) corresponds to the following formula:

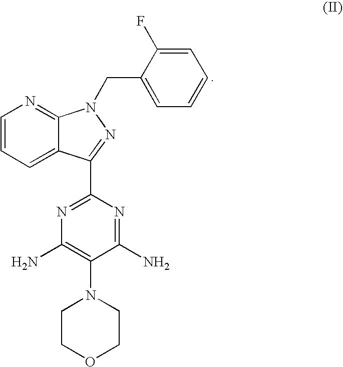

Compound (II), the preparation and use thereof as pharmaceutical have been disclosed in WO 00/06569.

Compound (III) corresponds to the following formula:

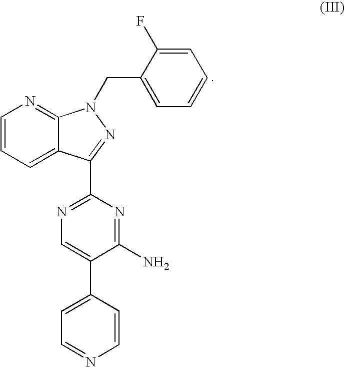

Compound (III), the preparation and use thereof as pharmaceutical have been disclosed in WO 00/06569 and WO 02/42301.

Compound (IV) corresponds to the following formula:

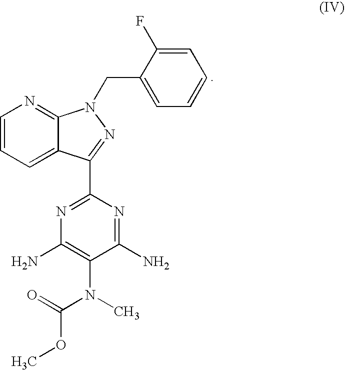

Compound (IV), the preparation and use thereof as pharmaceutical have been disclosed in WO 00/06569 and WO 03/095451.

Compound (IVa) corresponds to the following formula:

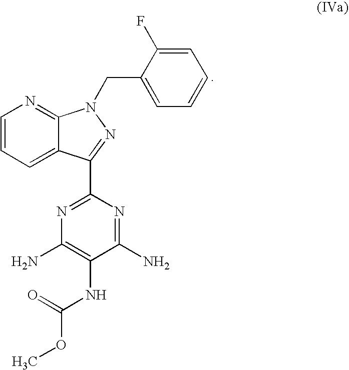

Compound (IVa), the preparation and use thereof as pharmaceutical have been disclosed in WO 00/06569 and WO 03/095451.

Compound (V) corresponds to the following formula:

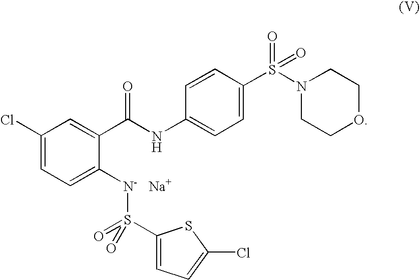

Compound (VI) corresponds to the following formula:

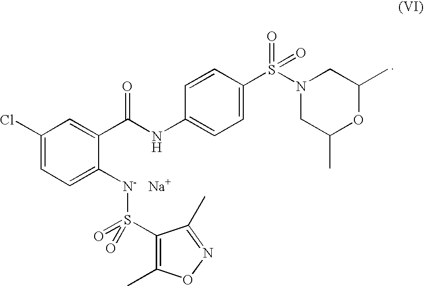

Compounds (V) and (VI), the preparation and use thereof as pharmaceutical have been disclosed in WO 00/02851.

COPD is usually induced by cigarette smoke as exogenous noxar. Genetic factors such as an α₁-antitrypsin deficiency or a bronchial hyperreactivity play a less important part. Inflammatory changes in the bronchial mucosa lead to damage to the airways and lung parenchyma. Chronic bronchitis, obstructive bronchiolitis and emphysema are the three pathological bases of the disorder which occur in varying severity in COPD and contribute to a progressive and expedited loss of the forced end-expiratory vital capacity (FeV₁).

Clinical signs are cough and expectoration for at least 3 months a year in at least two consecutive years. In addition, dyspnea occurs, initially during exercise, and later also at rest. Partial respiratory insufficiency is present, with an increase in the carbon dioxide concentration in the blood and later a global insufficiency with additional decline in the arterial oxygen concentration. Frequent exacerbations of COPD through bacterial infections, frequently with problem organisms, lead to an expedited reduction in the FeV₁.

Chronic hypoxia, inflammatory stimuli through nicotine and frequent bacterial exacerbations, and hyperinflation and overdistension of the airways through obstruction result in pulmonary vascular remodeling in COPD with intimal hyperplasia and medial hypertrophy. In cases of advanced COPD, average pulmonary arterial pressures above 40 mmHg are not uncommon, especially in patients with at least one episode of acute pulmonary failure. This is followed by chronic right-heart strain with the development of ankle edemas and chronic liver congestion, and an increasing deterioration in exercise capacity.

Besides long- and short-acting bronchodilatators (β-mimetics, anticholinergics, methylxanthines) to reduce the so-called dynamic hyperinflation COPD therapy also makes use of inhaled glucocorticoids to reduce the frequency of exacerbation and antibiotics in the case of bacterial bronchitis and pneumonias. However, the chronic progression of the disorder cannot be substantially influenced by any of the established therapy principles. Long-term oxygen therapy is recommended if there is global respiratory insufficiency with p_{A}O₂ below 55 mmHg. Applied consistently, this therapy improves the prognosis but cannot influence the remodeling of all the layers of the pulmonary arterial vessel walls.

There is as yet no approved therapy for treating COPD-associated pulmonary hypertension. Various systemic vasodilators such as, for example, calcium channel blockers have been tested in the past with disappointing results. Based on the analogy with idiopathic pulmonary arterial hypertension, more specific dilators with selectivity for the pulmonary circulation would be desirable, where appropriate with anti-remodeling properties and beneficial effects on the right heart hypertrophy.

It has surprisingly been found that the activators according to the invention of soluble guanylate cyclase listed below, compounds I-VI, are particularly suitable for producing pharmaceutical substances/medicaments for reducing pulmonary hypertension. Compared with the prior art, the compounds of the invention of the formulae I to VI have improved pharmacodynamic properties: on the one hand they act independently of NO produced endogenously in the pulmonary circulation, even if there is severe endothelial damage and the disease is at an advanced stage, to lower the pressure in the pulmonary arterial circulation. In addition, the stimulators of soluble guanylate cyclase enhance the effect of endogenously produced NO and, in this way, improve the gas exchange through a selective pulmonary vasodilatation of the ventilated areas, leading to a reduction in the intrapulmonary shunt with an increase in oxygenation.

The present invention relates to the use of compounds of the formulae (I-VI) and the salts, hydrates, hydrates of the salts thereof for the manufacture of a medicament for reducing pulmonary hypertension.

The present invention further relates to the use of compounds of the formulae (I-VI) and the salts, hydrates, hydrates of the salts thereof for the manufacture of a medicament for the treatment of acute and chronic lung disorders such as respiratory distress syndromes [acute lung injury (ALI), acute respiratory distress syndrome (ARDS)] and the treatment of COPD.

An additional exemplary embodiment of the present invention includes the procedure for the prophylaxis and/or for reducing pulmonary hypertension by use of at least one of the compounds of the formulae (I-VI).

The present invention further relates to pharmaceuticals comprising at least one compound of the invention and at least one or more further active ingredients, especially for the treatment and/or prophylaxis of the aforementioned disorders.

The compounds of the invention may have systemic and/or local effects. They can for this purpose be administered in a suitable way, such as, for example, by the oral, parenteral, pulmonary, nasal, sublingual, lingual, buccal, rectal, dermal, transdermal, conjunctival or optic route or as implant or stent.

The compounds of the invention can be administered in suitable administration forms for these administration routes.

Administration forms suitable for oral administration are those which function according to the state of the art and deliver the compounds of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, injection and infusion preparations in the form of solutions, suspensions, emulsions, lyophilizates or sterile powders.

Examples suitable for other administration routes are medicinal forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. anti-oxidants such as, for example, ascorbic acid), colors (e.g. inorganic pigments such as, for example, iron oxides) and masking tastes and/or odors.

The present invention further relates to pharmaceuticals which comprise at least one compound of the invention of the formulae (I-IV), normally together with one or more inert, non-toxic, pharmaceutically suitable excipients, and to the use thereof for the aforementioned purposes.

It has generally proved advantageous to administer amounts of about 0.01 to 5000 mg/kg, preferably about 0.5 to 1000 mg/kg, of body weight per day to achieve effective results.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The formulations can moreover comprise, appropriate for the intervention, active substance between 0.1 and 99% active ingredient, in a suitable manner 25-95% in the case of tablets and capsules and 1-50% in the case of liquid formulations, i.e. the active ingredient should be present in amounts sufficient to achieve the stated dose range.

### EXPERIMENTAL SECTION

### Hypoxia Model

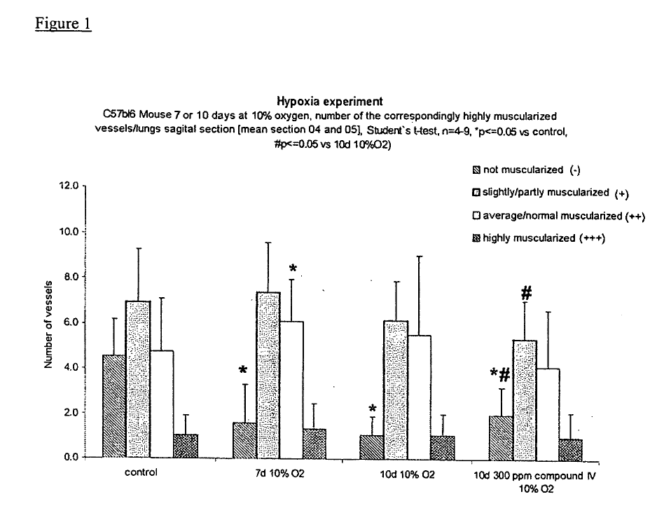

The method for investigating the effect of the compound (IV) of the invention in the model of experimental pulmonary hypertension was as described (Dumitrascu R, Weissmann N, Ghofrani H A, Beuerlein K, Schmidt H H H W, Stasch J P, Gnoth M J, Seeger W, Grimminger F, Schermuly R T, Activation of soluble guanylate cyclase reverses lung vascular remodeling and pulmonary hypertension evoked by hypoxia in mice, Circulation 2006, 113: 286-295). For this purpose, male C57B1/6J mice (Charles River Laboratories) were subjected to hypoxia (10% O₂) for 7 or 10 days. The control animals were kept in a normal oxygen environment. The mice were treated for 10 days with 300 ppm of compound (IV) in the feed. At the end of the test, the mice were sacrificed and the lungs were isolated. Histological workup and evaluation was carried out as in the abovementioned publication. Compared with the hypoxia control animals, the non-muscularized and partly muscularized fraction of the pulmonary vessels is significantly reduced in the mice treated with compound (IV) (FIG. 1).

1. The use of compounds of the formulae (I-VI) 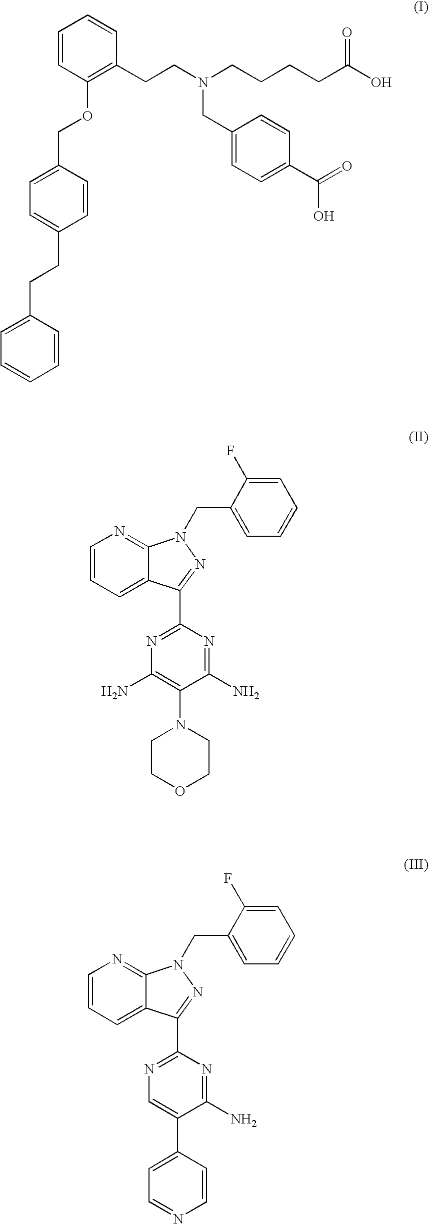 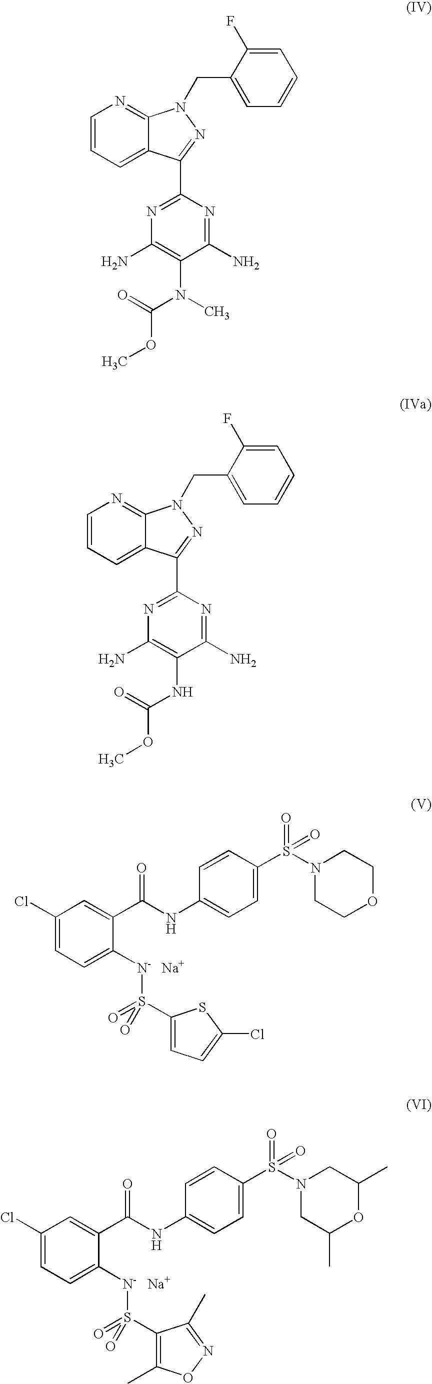
the salts, hydrates, hydrates of the salts thereof for the manufacture of a medicament for reducing pulmonary hypertension.

2. The use of compounds of the formulae (I-IV) and the salts, hydrates, hydrates of the salts thereof for the manufacture of a medicament for the treatment of acute and chronic lung disorders such as respiratory distress syndromes [acute lung injury (ALI), acute respiratory distress syndrome (ARDS)] and the treatment of COPD.

3. The use as claimed in either of claims 1 and 2, in which the medicament is used for an oral dosage form.

4. The use as claimed in either of claims 1 and 2, in which the medicament is given intravenously.

5. The use as claimed in either of claims 1 and 2, in which the medicament is used preventively.

6. A pharmaceutical composition for the treatment of pulmonary hypertension which comprises at least one substance as described in claim 1.

7. A pharmaceutical composition for the treatment of acute and chronic lung disorders such as respiratory distress syndromes [acute lung injury (ALI), acute respiratory distress syndrome (ARDS)] and the treatment of COPD which comprises at least one substance as described in claim 1.
